(19) United States
(12) Patent Application Publication
(10) Pub. No.: US 2021/0379223 A1
(43) Pub. Date: Dec. 9, 2021

(54) DEPLOYABLE ULTRAVIOLET SANITATION SYSTEM FOR AN AIRCRAFT

(71) Applicant: ASTRONICS PECO, INC., Clackamas, OR (US)

(72) Inventors: Paul TRESE, Wilsonville, OR (US); Robin Christensen, Aloha, OR (US); Chandelle Stanton, Gladstone, OR (US)

(73) Assignee: ASTRONICS PECO, INC., Clackamas, OR (US)

(21) Appl. No.: 17/343,322

(22) Filed: Jun. 9, 2021

Related U.S. Application Data
(60) Provisional application No. 63/036,695, filed on Jun. 9, 2020.

(51) Int. Cl. A61L 2/10; B64D 11/00

(52) U.S. Cl. CPC A61L 2/10; A61L 2202/25; B64D 11/00

(57) ABSTRACT

A sanitation system includes an overhead passenger service unit panel with a concealed light source providing a single wavelength of Ultraviolet light wherein the light source is deployed and sanitation is performed when the aircraft cabin is free of commercial passengers.

### CROSS-REFERENCE TO RELATED APPLICATION

This application claims the benefit of U.S. Provisional Application, Ser. No. 63/036,695, filed on 9 Jun. 2020. This U.S. Provisional application is hereby incorporated by reference herein in its entirety and are made a part hereof, including but not limited to those portions which specifically appear hereinafter.

### FIELD OF THE INVENTION

The invention is directed to an Ultraviolet sterilization system concealed in a passenger service unit of an aircraft.

### BACKGROUND OF THE INVENTION

Commercial aircraft cabins are well known for having a crowded mixture of passengers of varying health and hygiene. Concerns exist about cleanliness of the cabin surfaces from contagions, germs and other contaminants found on surfaces passengers interact with.

A need therefore exists to sanitize surfaces of a commercial aircraft to provide the passengers an increased level of cabin cleanliness while avoiding many current labor intensive practices that expose sanitation workers to such contaminants and increase turnaround times.

### SUMMARY OF THE INVENTION

Commercial aircraft cabins are comprised of a number of material and surface treatments. During any flight passengers routinely interact with sidewalls, seating surface, seatbelts, seat backs, In Flight Entertainment (IFE) systems and tray tables. Each of these surfaces provide a potential source of contamination. Sanitizing these surfaces can be a time-consuming effort, requiring application and removal of cleaning agents between flights.

Located above aircraft seating is generally a Passenger Service Unit (PSU) which contains reading lights, Personal Air Outlets (PAO) or “gaspers” and emergency oxygen systems as well as miscellaneous mandatory or optional signage. The present invention utilizes this system of panels to provide a deployable location to install a sanitation system at each seat grouping.

Ultraviolet light has long been used to sanitize surfaces in hospitals, water sources, humidifiers and other surfaces requiring a high level of sanitation. Ultraviolet light in the UV-C wavelength has been demonstrated effective against a wide range of viruses and bacteria. It is also widely understood that Ultraviolet Light in the UV-C range can be harmful to humans when exposed. Skin cancers and cataracts of the eye are associates with exposure to UV-C light.

According to a preferred embodiment of this invention, a UV-C light source is concealed in the PSU Panel that extends above the passenger seats, and is preferably stowed while the aircraft is occupied by passengers, flight crew or a ground personnel. When the aircraft is unoccupied, an Ultraviolet light system above each seat or set of seats is deployed to sanitize all surfaces that are exposed to the UV-C light source. A desired duration of the UV saturation is a function of the distance from the light source to the surfaces to be sanitized. This system preferably utilizes an LED light source emitting a single wavelength of UV-C light. That single wavelength of light in the UV-C band can be from 200 nm to 280 nm.

### BRIEF DESCRIPTION OF THE DRAWINGS

FIG. 1 illustrates the mechanical system associated with a panel located in the cabin of an aircraft which contains the deployable Ultraviolet panel;

FIG. 2 shows a view of the Ultraviolet system panel in a deployed state; and

FIG. 3 shows a view of the Ultraviolet system in a stowed state.

### DETAILED DESCRIPTION OF THE INVENTION

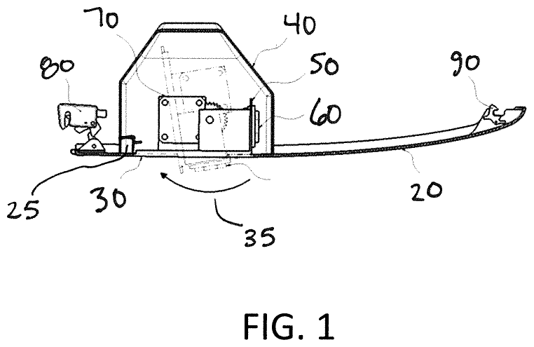

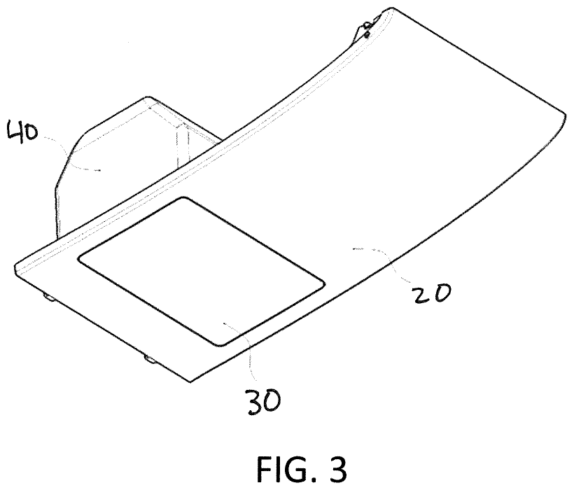

The present invention provides a system and method for sanitizing the surfaces which the passenger interacts with in a commercial aircraft cabin. FIGS. 1-3 show the Ultraviolet Light panel which is typically located above the passenger seats, but can be installed in other locations throughout the aircraft as needed. Applicable areas could include lavatories, galley area, flight attendant seating areas, entryways, crew rest areas and aisleways. The Ultraviolet light panel is preferably concealed while in flight and will only be deployed when persons are clear of the immediate area of the light. Safety features to prevent inadvertent deployment can also be integrated into the system as an added level of safety.

According to one preferred embodiment of the invention, the Ultraviolet light panel may be positioned above each and every seat on the aircraft and then may be deployed during aircraft turnaround and/or routine maintenance intervals. According to another preferred embodiment of the invention, the Ultraviolet light panel may be positioned over groupings of 2 or 3 or more seats and may be deployed and then manually or automatically rotated to sweep across each seat area in a predefined increment or interval. Whether the Ultraviolet light panel is assigned to a single seat or a series of seats, the focus of the light is preferably adjustable and moveable to ensure coverage over all seat areas.

As shown in FIGS. 1-3, a sanitation system comprises an overhead passenger service unit panel 20 including a concealed light source providing a single wavelength of Ultraviolet light wherein the light source is deployed and sanitation is performed when the aircraft cabin is free of commercial passengers.

In particular, according to one embodiment, the panel 20 replaces a spacer panel that is generally found in the ceilings of commercial aircraft. The panel 20 is preferably attachable to aircraft using an outboard rail latch 80 and an inboard rail latch 90 that affix to existing rails within the aircraft cabin structure.

A door 30 is preferably integrated with the panel 20. The door 30 is desirably flush with the panel 20 while in a stowed condition, that is, while the sanitation system is not deployed. The door 30 preferably accommodates a UV housing 50.

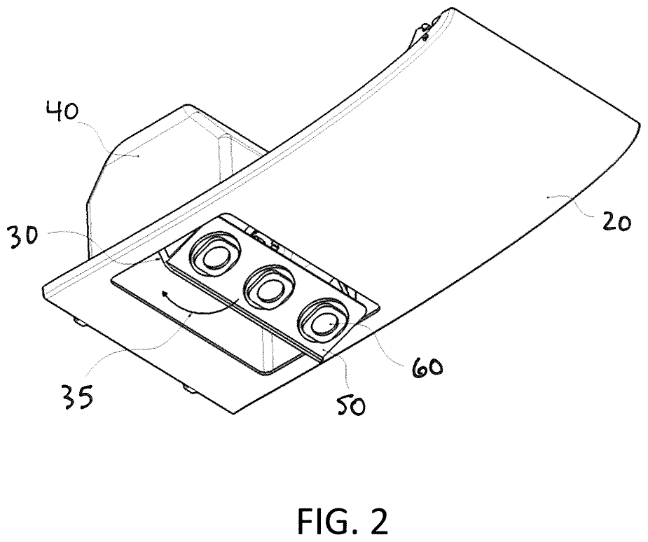

As shown in FIGS. 1 and 2, the UV housing 50 preferably includes three UV light modules 60 that together comprise the light source used for sanitizing the aircraft cabin. Although three UV light modules 60 are shown, more or fewer UV light modules 60 may be adapted to the sanitation system described based upon factors such as distance from the surfaces to be disinfected, the UV bandwidth desired, the duration of sanitization cycles desired, and other factors. The at least three UV light modules 60 are preferably arranged in a row and are configured to sweep across a set of one, two or three or more seats.

A drive motor 70 is preferably positioned within the panel 20 and is configured to rotate the UV housing 50 and attached UV light module(s) 60 through a predetermined sweep. The drive motor 70 is preferably powered by a 12 VDC power supply. The drive motor 70 is preferably configured with a controller to rotate in one or more desirable routines, as described in more detail below.

Preferred UV light modules 60 include a single wavelength of light in the UV-C band. A preferred bandwidth is in a range of 200 nm to 280 nm, most desirably approximately 270 nm.

The sanitation system and panel 20 as described preferably additionally include a drip shield 40 positioned around the UV light module 60. Condensation may accumulate in the interstitial portions of the aircraft above the cabin infrastructure and such moisture may otherwise create issues for the sanitation system mechanicals and UV light modules 60 if not properly guarded.

A corresponding method of sanitizing an aircraft cabin having a plurality of seats includes positioning an overhead passenger service unit panel 20 over one or more seats, preferably one panel 20 per seating group; integrating a door 30 with the panel 20 to rotate between a stowed position generally flush with the panel 20 and a deployed position; positioning a plurality of UV light modules 60 with respect to the door 30, the UV light modules 60 emitting UV light in a single wavelength in the UV-C band; configuring the door 30 to deploy when the aircraft cabin is empty; and activating the light modules 60 in a single bandwidth between 200 nm and 280 nm.

The door 30 may be preferably deployed to oscillate in a continuous back-and-forth sweep across the two or more seats. Alternatively, or in addition, the door 30 may be deployed such that rotation of the door 30 is paused for a predetermined time period generally in alignment with each seat. The duration of efficacy may range between approximately 80 seconds to 30 minutes, depending on factors such as bandwidth, distance to surfaces, surface materials, and inherent cleanliness of surfaces. The door 30 is preferably configured to rotate in a direction perpendicular to an alignment of the plurality of seats.

To safely deploy and activate the sanitation system, a lock 25 may be activated on the door 30 at all times when the aircraft cabin is occupied. In such a way, the sanitation system and/or the door 30 may be configured to open only when each of the plurality of seats is unoccupied. The system may be additionally integrated with the cabin management system of the aircraft to deploy and/or stow the UV housing 50 during intended and desired increments thereby eliminating passenger exposure to UV light and damage to the system by passengers.

Preferably the sanitation operation would take place during change over and before or after the cabin cleaning crew has addressed the cabin.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A sanitation system comprising:
an overhead passenger service unit panel including a concealed light source providing a single wavelength of Ultraviolet light wherein the light source is deployed and sanitation is performed when the aircraft cabin is free of commercial passengers.

2. The sanitation system of claim 1 wherein the single wavelength of light is in the UV-C band.

3. The sanitation system of claim 1 wherein the UV-C band is in a range of 200 nm to 280 nm.

4. The sanitation system of claim 1 further comprising:
a door integrated with the panel, the door accommodating a UV housing.

5. The sanitation system of claim 4 further comprising a drive motor, the drive motor configured to rotate the UV housing through a predetermined sweep.

6. The sanitation system of claim 5 wherein the UV housing includes at least three UV light modules comprising the light source.

7. The sanitation system of claim 6 wherein the at least three UV light modules are arranged in a row and are configured to sweep across a set of two or more seats.

8. The sanitation system of claim 1 further comprising a drip shield positioned around the UV light module.

9. The sanitation system of claim 1 wherein the light source comprises a plurality of UV-C lights emitting light in a range of approximately 200 nm to 280 nm.

10. A sanitation system for an aircraft cabin having a plurality of seats, the sanitation system comprising:
an overhead passenger service unit panel;
a door integrated with the panel, the door configured to rotate between a stowed position generally flush with the panel and a deployed position;
a UV light housing connected with respect to the door;
a plurality of UV light modules connected with respect to the UV light housing, the UV light modules emitting UV light in a single wavelength in the UV-C band.

11. The sanitation system of claim 10 wherein the UV-C band is in a range of 200 nm to 280 nm.

12. The sanitation system of claim 10 further comprising a drive motor, the drive motor configured to rotate the UV housing through a predetermined sweep.

13. The sanitation system of claim 10 wherein the UV housing includes at least three UV light modules comprising the light source.

14. The sanitation system of claim 10 wherein the door is configured to open only when each of the plurality of seats is unoccupied.

15. The sanitation system of claim 10 wherein the door is configured to rotate in a direction perpendicular to an alignment of the plurality of seats.

16. The sanitation system of claim 10 wherein the door is configured to rotate across a group of two or more seats.

17. A method of sanitizing an aircraft cabin having a plurality of seats, the method comprising:
positioning an overhead passenger service unit panel over two or more seats;
integrating a door with the panel to rotate between a stowed position generally flush with the panel and a deployed position;
positioning a plurality of UV light modules with respect to the door, the UV light modules emitting UV light in a single wavelength in the UV-C band;
configuring the door to deploy when the aircraft cabin is empty; and activating the light modules in a single bandwidth between 200 nm and 280 nm.

18. The method of claim 17 further comprising oscillating the door in a continuous back-and-forth sweep across the two or more seats.

19. The method of claim 17 further comprising pausing rotation of the door for a predetermined time period in a direction of each seat.

20. The method of claim 17 further comprising:
activating a lock on the door when the aircraft cabin is occupied.
